(19) United States
(12) Patent Application Publication
(10) Pub. No.: US 2026/0249108 A1
(43) Pub. Date: Aug. 27, 2026

(54) METHOD OF TREATMENT FOLLOWING ORGAN TRANSPLANT WITH ACCOUSTIC ENERGY

(71) Applicant: CURESPEC LTD., Yehud (IL)

(72) Inventors: Avner SPECTOR, Yehud (IL); Rotem KAYNAN, Kibbutz Barkai (IL); Hagay SPECTOR, Yehud (IL)

(21) Appl. No.: 19/540,800

(22) Filed: Feb. 16, 2026

Related U.S. Application Data
(60) Provisional application No. 63/759,458, filed on Feb. 17, 2025.

(51) Int. Cl. A61N 7/02; A61H 23/00; A61N 7/00

(52) U.S. Cl. CPC A61N 7/02; A61H 23/008; A61H 2205/083; A61N 2007/0004

(57) ABSTRACT

A method of treatment of a transplanted organ with noninvasive acoustic energy to treat and/or prevent Delayed Graft Function (DGF).

### RELATED APPLICATIONS

The present disclosure claims benefit of and priority to U.S. provisional patent application no. 63/759,458, filed February 17, 2025, the disclosure of which is herein incorporated by reference in its entirety.

### FIELD OF THE INVENTION

The present invention relates to a method of treatment of a human or animal transplanted organ, following an organ transplant and in particular the application of acoustic energy following the organ transplant to treat and/or to alleviate the onset of Delayed Graft Function (DGF).

### BACKGROUND OF THE INVENTION

Acoustic energy has been used in medicine for various purposes including imaging such as ultrasound imaging. Acoustic energy has also been utilized as a medium for conveying or transmitting energy to a remote treatment sites, for example, as respectively accomplished with ultrasound or shockwaves.

One form of therapeutic acoustic energy is Extracorporeal Shock Wave Therapy (herein referred to as ‘ESWT’) which is a non-surgical, non-invasive treatment of medical conditions using acoustic shockwaves. The first use of shockwave therapy in the early 1980’s was utilized to fragment kidney stones termed shockwave lithotripsy. Continued development of shockwave treatment showed the possibility of stimulating bone formation, angiogenesis, as well as other orthopedic indications. However, medical literature suggests that lithotripsy creates hypertension and some damage to the kidney including hematuria during the procedure.

A shockwave is a form of acoustic energy resulting from phenomena that create a sudden intense change in pressure, for example an explosion or lightning. The intense changes in pressure produce strong waves of energy that can travel through any elastic medium such as air, water, human soft tissue, or certain solid substances such as bone.

Shockwaves are characterized by the delivery of a sequence of transient pressure disturbances characterized by an initial high peak pressure with a fast pressure rise followed by rapid wave propagation with diminishing amplitude over its lifecycle. Such that shockwaves characteristically have a quick lifecycle, starting with a big high amplitude pressure peak followed by a gradual diminishing pressure amplitude having amplitude of about 10-20% of the initial pressure peak. Shockwave are further characterized in that they do not produce heat within the tissue.

Acoustic shockwaves are primarily generated by three different methods, electrohydraulic (also referred to as spark gap), electromagnetic (also referred to as ‘EMSE’), and piezoelectric. Each method needs an apparatus to focus the generated shockwave so as to provide a focal point and/ or focal zone for the treatment area. In the focal zone shockwaves produce much higher pressure impulses as compared with the zones outside of the focal zone.

Traditionally shockwaves have been used in medicine as a noninvasive means for treating a variety of anomalies such as kidney stones (lithotripsy), fragmentation of calcification, chronic orthopedic inflammation healing, bone healing (osteogenesis), wound healing, revascularization, angiogenesis are well known and described in medical literature.

US Patent No. 7,507,213 to Schultheiss, et al. discusses invasive stimulation of kidney by surgically exposing the organ for example heart or kidney prior to applying shockwave therapy.

US Patent Publication No. 2011/0257523 to Hastings et al. discusses a method utilizing high intensity focused ultrasound (HIFU) for ablating innervated tissue of the kidney, for denervating renal vasculature, including disruption and termination of renal sympathetic nerve activity, to improve cardiac and/or renal function particularly that associated with hypertension.

Traditional ESWT utilizes high energy shockwave, for example in the form of lithotripsy, to evoke high pressure shockwave with an energy density of about 0.6-1.1 mJ/mm². The application of low energy shockwave treatment (herein 'LESW') has been described by Krause US Patent No. 5,545,124 and Warlick in US Patent Publication No. 2007/0142753 for the treatment of pain and pancreatic tissue regeneration, respectively.

### SUMMARY OF THE INVENTION

Delayed Graft Function (herein ‘DGF’) is a complication following organ transplant and invasive procedures such as open heart surgery. DGF following transplantation has been widely reported both in kidney transplant (Moreso et al. 1999. Nephrol. Dial. Transplant. 14(4):930-35; DOI: 10.1093/ndt/14.4.930); and liver transplant (Kuchipudi et al. 2015, Am. J.of Gastroenterology 110:p S855, October 2015), however, may occur following any major organ transplant procedure such as kidney, liver, heart, lung, or any such double transplantation procedure. Complications of DGF can lead to the loss of the transplanted organ, a treatment thereof is not provided in the state of the art.

There is an unmet need for, and it would be highly useful to have, a noninvasive method for the treatment of DGF in transplanted organs and in particular to such organs that are sourced and/or harvested from the deceased.

Embodiments of the present invention provide a method for treating and/or preventing DGF following an organ transplant procedure. In particular, following an organ transplant selected from kidney transplant, liver transplant, heart transplant, skin graft, vascular graft, graft, lung transplant, multiple transplant procedure of two or more organs, the like procedure or organ transplant of the human or animal body.

Embodiments of the present invention provide a treatment to the transplanted organ following the transplant procedure, in particular by treating the implanted organ with acoustic energy, most preferably low energy extracorporeal shockwaves so as to achieve an immediate and/or short term effect of vasodilation in the tissue surrounding the implanted organ.

In embodiments, the initial treatment may be provided immediately following the transplant procedures within 24 hours following the transplant procedure.

In embodiments, the initial treatment may be provided following the transplant procedures and within 24 hours after the initial indications and/or emergence and/or development of DGF.

In embodiments, the initial treatment may be provided within a 48 hour window following the transplant procedure.

In embodiments, the initial treatment is preferably provided immediately following the transplant procedures within a time frame of from 2 hours and up to about 8 hours following the organ transplant procedure.

In embodiments a long term effect is further achieved with the treatment according to embodiments of the present invention, wherein the treatment with acoustic energy, most preferably low intensity shockwave to the transplanted organ further provides for triggering a cascade of biomolecular activity involving a plurality of biomolecular factors, to improve the healing process and return to function of the implanted organ.

In embodiments of the present invention the low energy shockwave treatment is provided to at least a portion of an implanted organ to treat DGF provides both an immediate effect comprising vasodilation after administration of acoustic energy, most preferably in the form of low intensity shockwave to the tissue , and a long term effect comprising triggering a cascade of biomolecular activity in the implanted organ so as to improve the organ functionality, by enhancing angiogenesis and anti-inflammatory responses, increasing tissue perfusion, and preventing or reducing the development of excess scar tissue and/or fibrosis in and around the treatment area.

In embodiments, the method of treatment of the present invention comprises the application of acoustic energy, most preferably in the form of low energy shockwave, following an organ transplant. The application of acoustic energy is intended to be a part of a standard of care and/or treatment protocol following implantation, at least for an implanted kidney, so as to prevent the onset of DGF which is currently exhibited in about 60% of cases following a transplant procedure.

In embodiments of the present invention the treatment provides for reducing the time it takes the implanted organ to return to function, which is particularly evident in the case of kidney transplant, and particularly when the origin of the implanted kidney is from a deceased individual. Accordingly, the treatment protocol according to embodiments of this invention improves the probability and the effectiveness of the kidney transplant process. One of the results of this method of treatment according to embodiments of the present invention is that the collateral blood vessels of the implanted organ are revived, that would otherwise (if not treated according to the present invention), would not function after the kidney transplant, due to the onset of DGF. Accordingly, a quicker return to function of the implanted organ is due to improved vasodilation of the implanted organ as a result of the treatment regimen following implantation and improve recovery time so as to reduce the length of stay in hospital and the transplant success rate. In embodiments the method of the present invention provides for reducing the time to return to function of an implanted organ from at least two weeks (without treatment) to about three days (with treatment).

Furthermore, the method of treatment according to embodiments of the present invention may further provide a long term effect in an implanted organ, for example in at least an implanted human kidney, as the treatment according to embodiments provides for triggering a cascade of molecular activity that is garnered to control Extra Cellular Matrix ('ECM') remodeling, in and around the treatment area, namely the implanted organ. Preferably ECM remodeling is provided by the delivery acoustic energy, most preferably in the form of low energy shockwave at the treatment site comprising the transplanted organ, that in turn elicit a cascade of bio-molecular activity that controls the expression of at least one or more of: biomolecular factors, growth factors, ECM factors and/or ECM associated factors at the treatment site so as to allow for both vasodilation and for non-fibrotic tissue remodeling at the treatment site.

In embodiments, the low energy shockwave treatment preferably provides for triggering a cascade of bio-molecular activity involving at least one growth factor and/ or biomolecular factor selected from the group for example including but not limited to eNOS, VGF, TGFβ; TIMP-1, FAK, SCF, HGF, FAK, or any combination thereof.

In embodiments, the treatment protocol of the present invention comprises an active treatment period wherein acoustic energy, preferably in the form of low energy shockwaves, is delivered to the transplanted organ, as soon as possible after the transplant procedure, most preferably at least 2 hours after transplantation as a precautionary measure to avoid the onset of DGF, which tends to occur in about 60% of current transplant procedures (from deceased doners).

In embodiments, the treatment protocol may also be initiated once DGF has been detected, for example for the kidney when urine output is less than 0.005 liter at 4 hours post transplantation, and/or creatinine levels are high at 8 hours post-transplant procedure.

In embodiments, the active treatment period may comprise providing acoustic energy to the implanted organ and more preferably low energy shockwaves for a period of at least once a day for a period of at least 21 day or until such a time that the implanted organ recovers and functions properly or shows signs of overcoming DGF.

In embodiments the treatment may be provided daily where there are up to three individual treatment sessions per day.

In embodiments the low energy shockwave treatment is administered to the implanted organ over at least one treatment area and/or at least one or more treatment zones defined about the organ.

In embodiments, the treatment area, comprising the implanted organ, may be divided into a plurality of individual treatment zones and/ focal zones.

In embodiments, the treatment area may be divided into a plurality of smaller treatment zones and/or focal zones based on a number of parameters for example including but not limited to treatment applicator head, size of treatment area the like or any combination thereof.

In embodiments each active treatment session may include at least 100 and up to about 500 low energy shockwaves that are administered over a single treatment focal zone, for example having a diameter of about 2 mm (millimeters) and up to about 50 mm (millimeters). Optionally each active treatment session includes up to about 5000 low energy shockwaves that are administered over a treatment area.

In embodiments the low energy shockwave treatment may be administered to a transplanted organ, for example including but not limited to at least one or more of kidney, heart, liver, lung, graft, vascular graft, and/or skin graft that may be exhibiting signs of DGF.

In embodiments an initial treatment may include at least two and up to 20 treatment sessions that may be provided immediately following the organ transplant procedure, and most preferably initiated within a time frame of at least 2 hours post procedure.

For example, a treatment protocol of up to four sessions may be provided immediately following the organ transplant procedure wherein each session consisting of at least 100 and up to 5000 shockwaves. Optionally, the treatment may be initiated with the first indication and/or signs of DGF is identified.

Optionally treatment protocol parameters may for example include but is not limited to the number of treatments sessions, the duration of a treatment protocol, timing of active and/or inactive treatment sessions, frequency of session, any combination thereof or the like.

In embodiments, the number of active treatment sessions may be provided from about 1 session and up to about 40 sessions. Optionally 12 active treatments may be provided during the treatment protocol according to the present invention. Optionally the number of active treatment session may for example be 1, or 2, or 3, or 4 or 5 or 6, or 7 or 8 or 9 or 10 or 11, or 12 or 13 or 14 or 15 or 16 or 17 or 18 or 19 or 20 or so sessions.

Optionally the duration of the treatment protocol according to the present invention may be from about 1 day up to about 4 weeks or the equivalent of 1 day up to about 28 days.

In embodiments treatment may be provided periodically, continuously, sequentially, intermittently, according to a schedule comprising consecutive treatment sessions and/or with at least one or more intersession recesses and/or rest periods. Optionally the length of the recesses and/or rest period may vary according to the required treatment protocol.

In embodiments acoustic energy treatment to an implanted organ may be provided in the form of shockwave or ultrasound pulses based on parameters that may for example include but are not limited to number of shockwave and/or ultrasound pulses s, frequency of shockwaves and intensity of the shockwave, or the like.

The shockwave intensity utilized according to embodiment of the present invention may be provided from about 0.02 mJ/mm² to about 0.3 mJ/mm². Optionally and preferably shockwave intensity may be provided from about 0.09mJ/mm² to about 0.11mJ/mm². Optionally and more preferably shockwave intensity may be provided at about 0.1mJ/mm².

Optionally shockwave pressure utilized in embodiments of the present invention may for example be from about 30 atm to about 200 atm and/or from about 3Mpa to about 20Mpa.

Optionally shockwave frequency may be provided from about 60 shockwaves per minute to about 360 shockwaves per minute. Optionally and preferably a shockwave frequency may be provided from about 120 shockwaves per minute to about 240 shockwaves per minute. Optionally and most preferably a shockwave frequency may be provided at about 180 shockwaves per minute.

Optionally shockwave frequency may be provided from about 1Hz to about 6Hz. Optionally and preferably a shockwave frequency may be provided at about 2-4Hz. Optionally and preferably a shockwave frequency may be provided at about 2Hz.

Optionally the number of shockwaves per treatment session may be provided from about 100 shockwave up to about 5000 shockwaves. Optionally about 1800 shockwaves per session may be provided.

In embodiments the method of treatment may a combined acoustic energy both in the form of ultrasound waves and/or shockwaves. Optionally within a treatment session a combined use acoustic energy including both ultrasound and shockwaves.

In embodiments the method of treatment may be combined acoustic energy both in the form of ultrasound waves and/or shockwaves in an alternating regime wherein in ultrasound energy and shockwave energy are changed in alternate sessions.

Embodiments of the present invention provide a treatment of an implanted organ and/or tissue with low energy shockwaves, that are provided to bring about vasodilation of the tissue associated with the transplanted organ and to further improve and/or increase blood flow in the transplanted organ for example including but not limited to the kidney, lung, liver, heart, graft, vascular graft, skin graft, or the like.

In embodiments, the low energy acoustic energy preferably in the form of shockwaves and/or optionally in the form of ultrasound waves having parameters selected form at least one or more of: a frequency of about 0.4 MHz up to about 3 MHz, energy density from about 100 mW/cm² up to about 800 mW/cm² SATA, duty cycle of about 10% up to about 44% , treatment exposure time per treatment zone selected from 0.5 minutes up to about 10 minutes, the like or any combination thereof. The acoustic energy is preferably delivered to at least a portion of a the implanted organ during a treatment session according to the present invention may be applied to at least one or more treatment zone. Optionally a treatment area along the implanted organ for example may include but is not limited to at least one selected from the kidney, heart, liver, lung, graft, vascular graft and/or skin graft or the like . In embodiments the implanted organ may be divided into a plurality of individual treatment zones. Optionally the number of treatment zones utilized may be determined according to the size of the treatment area. Optionally the number of treatment zones utilized may be determined according to at least one or more parameter associated with the shockwave generating device and may for example include but is not limited to the shockwave device treatment head and its effective treatment zone capabilities. For example, a treatment area may be divided into a plurality of smaller treatment zones, from about 1 up to about 15 treatment zones, comprising the implanted organ.

Optionally the overall number of shockwaves delivered to a treatment area may be distributed about a plurality of treatment zones in any manner required for the treatment, for example including evenly distributing the number of shockwaves based on the number of zones, or by unevenly distributing the number of shockwaves per zones, or the distribution of shockwaves may be based on the underlying tissues within the individual treatment zones, the like or any combination thereof. For example, a plurality of zones from about 5 to about 15 zones may be treated with 100 shockwaves to about 500 shockwaves within a treatment session to provide for a treatment protocol including up to about 5000 shockwaves delivered to the implanted organ.

Optionally the shockwave treatment according to the present invention may be applied to at least a portion of the transplanted organ from at least one or more optional approaches for example including but not limited to prone, lateral, supine, or any combination thereof, providing for appropriate non-invasive access to the portion of the implanted organ.

Within the context of this application the terms aqueous solution, aqueous medium, or aqueous environment may be used interchangeably to refer to an enclosure, opening, lumen, or space that is placed in an aqueous solution or mixture for example including but not limited to water, medicated water, ionized water, oil, gel, treated water or the like solution or mixture in a liquid state.

Within the context of this application the term extracorporeal shockwave therapy (‘ESWT’) refers to shockwave therapy provided with all forms of shockwave generating device.

Within the context of this application the term low energy extracorporeal shockwave therapy (“LESW”) or low energy shockwave therapy or low intensity shockwave therapy may interchangeably refer to shockwave therapy provided with all forms of shockwave generating device and providing an energy density of up to about 0.30 mJ/mm².

Within the context of this application the term molecular factor or biomolecular factor refer to any one or more factors and/or its conformation or subunit thereof, for example including but not limited to mRNA, protein, molecule, receptor, transcription factor, enzyme, inhibitor, promotor, activator, repressor, regulating factor, protein, hormone, growth factor, chemokine, cytokine, kinase, transmembrane protein, membrane protein, stem cell, progenitor cell, or the like.

Within the context of this application, unless indicated otherwise, the term "molecular factor" or "biomolecular factor" may refer to any one or more of the following factors known in art: Transforming Growth Factor beta ('TGF-β' or 'TGFbeta'); Tissue Inhibitor of MetalloProteinases (TIMP); TIMP metallopeptidase inhibitor 1 (TIMP-1); Focal Adhesion Kinase ('FAK'); Stem Cell Factor ('SCF'); Hepatocyte Growth Factor ('HGF'); Stromal Derived Cell Factor 1 ('STF-1'); Octamer-Binding Transcription Factor 4 (Oct-4); Kidney Injury Molecule-1 (KIM-1); Stromal Cell-Derived Factor 1 (SDF-1) also known as C-X-C motif chemokine 12 (CXCL12); Hypoxia Inducible Factor 1 ('HIF-1'); Norepinephrine (NE); Integrin; Beta 1 integrin; Monocyte Chemoattractant Protein-1 ('MCP-1'); mRNA of the cluster of differentiation 3 T-cell co-receptor ('CD3 mRNA'); Vascular Endothelial Growth Factor (VEGF); Fibroblast Growth Factors (FGF); Endothelial Nitric Oxide Synthase (eNOS); Angiopoietins (Ang); platelet-derived growth factor; angiogenin; angiotropin; hepatocyte growth factor; platelet endothelial cell adhesion molecule; angiostatin; endostatin; thrombospondin; Chemokine family of the form CXC ('CXC'); Nitric Oxide ('NO'); Nuclear Factor Kappa-light-chain-enhancer of activated B cells ('NFkapaB'); Tumor Necrosis Factor Alpha mRNA ('TNF-alpha mRNA'); pigment epithelium, endothelial progenitor cell or the like.

Within the context of this application the term treatment area refers to an area of at least a portion of the transplanted organ, for example including but not limited to kidney, heart, liver, lung, graft, vascular graft and/or skin graft, that is being treated with low energy shockwaves. In embodiments, the treatment area may be of any size, shape, volume. Such a treatment area may be divided into a number of smaller treatment zones that herein may be interchangeably referred to as sub-treatment zones, individual treatment zones and/or focal zones. A treatment zone and/or focal zone may be any portion of the implanted organ having a diameter of about 2mm up and up to about 50 mm. A treatment area within the context of this application may receive up to about 5000 low energy shockwaves during a treatment session, while an individual treatment zone and/or focal zone may receive up to about 500 low energy shockwaves, during a treatment session.

Within the context of this application the term acute tissue trauma and/or acute tissue trauma event that occurs as a results of traumatic event may refer to any event affecting tissue causing acute tissue trauma. Such an acute tissue trauma event may for example include but is not limited to: medical intervention, blunt force trauma, lesion, invasive medical intervention, surgery, suture, myocardial infarction, acute kidney injury, hysterectomy, prostatectomy, biopsy, mastectomy, cancer treatment, chemotherapy, biological therapy, cell therapy, stem cell therapy, any combination thereof or the like.

Within the context of this application the term shockwave treatment device refers to a device comprising a controller and/or computer and a shockwave treatment applicator as is known in the art. For example, a shockwave treatment device comprises controller and/or computer that controls the shockwave treatment produced by the shockwave treatment applicator and/or treatment head.

Within the context of this application shockwave properties and/or parameters may be interchangeably represented in different units of measure as is accepted in the art to refer to the same and/or equivalent units of measure. For example, shockwave pressure may be interchangeably provided in units of atmospheres (“atm”) or Pascals (‘pa’) or mega Pascals (Mpa). Shockwave frequency may be provided in relative of absolute units, for example including but not limited to hertz (“Hz”) and/or shockwaves per unit time, shockwave per minute, or the like.

Within the context of this application the term renal and/or kidney structures refers to any of the following structures nephron, glomerulus, Bowman’s capsule, tubules, medulla, renal artery, renal vein, renal pelvis, papilla, adrenal glands, adrenal cortex, adrenal medulla, phrenic arteries, and adrenal vein, neural tissue directly or indirectly innervating the kidney and renal structures, kidney neural system including renal sympathetic and renal para-sympathetic nerves, renal sympathetic nerves that lie within and immediately adjacent to the wall of the renal arteries.

Within the context of this application the term transplanted organ refers to any one or portion of any part, cell, tissue, system associated with the implanted organ anatomy, for example including but not limited to heart, lung, kidney, liver, gall bladder, pancreas, spleen, stomach, intestine, gastrointestinal tract, lymphatic system, skeletal muscles, smooth muscles, cardiovascular system, urinary bladder, skin, female reproductive system, uterus, ovaries, fallopian tubes, cervix, male reproductive system, penis, vas deferens, testicles, prostate, or any portion, cell, tissue or functional group of the kidney anatomy. The kidney human or portion thereof refers to any part, cell, tissue, system, or organ having in-vivo origin, in-vitro origin, as a result of a transplant, the like or any combination thereof.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

### In the drawings

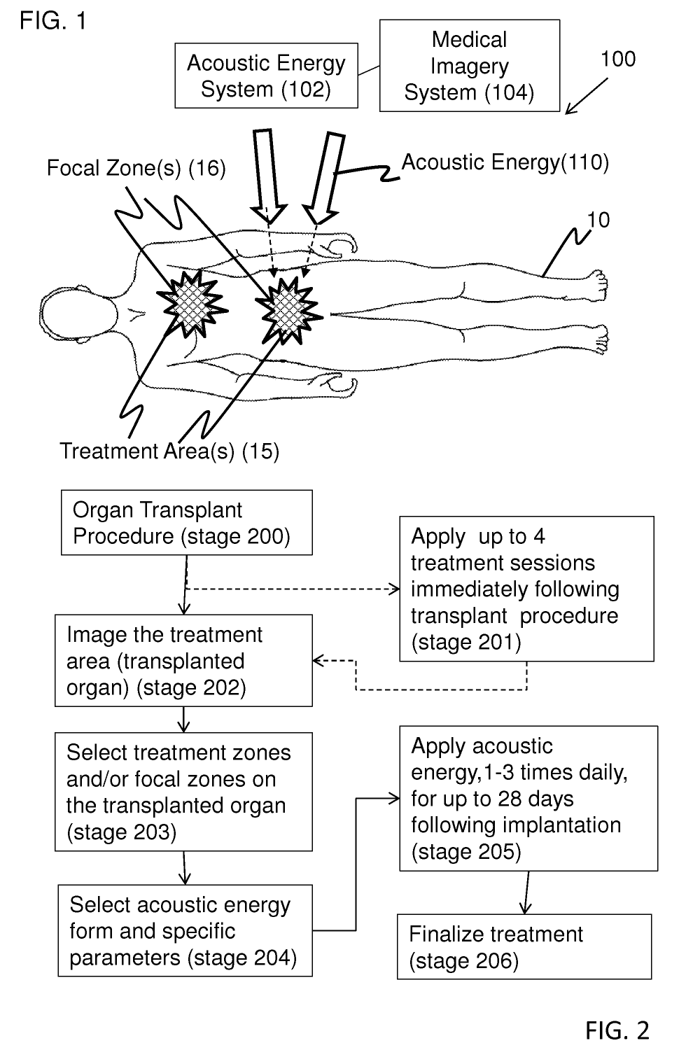

FIG. 1 is a schematic illustrative diagram of a portion of the human anatomy receiving the low energy shockwave treatment to an implanted organ so as to avoid or treat Delayed Graft Function according to embodiments of the present invention; and

FIG. 2 is a flowchart of depicting the method of treatment according to embodiments of the present invention; and

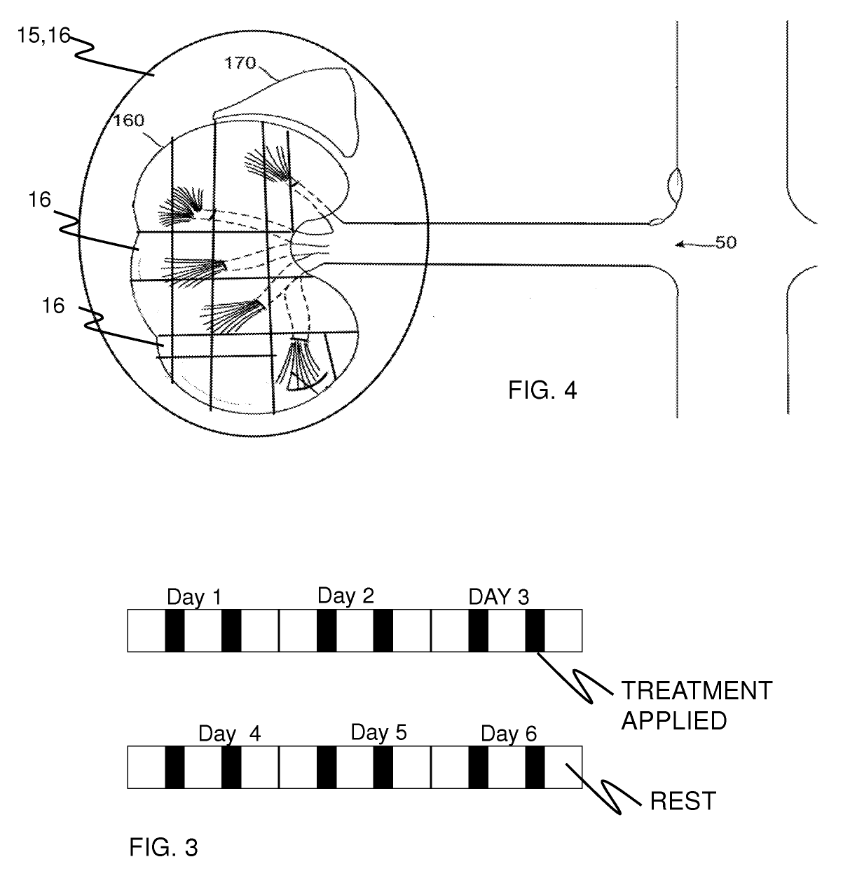

FIG. 3 is a schematic illustrative diagram of a Gantt chart of an optional treatment protocol according to optional embodiments the present invention; and

FIG. 4 is a schematic illustration of the treatment site and focal zones according to embodiments of the present invention.

### DESCRIPTION OF THE PREFERRED EMBODIMENTS

The principles and operation of the present invention may be better understood with reference to the drawings and the accompanying description.

FIG. 1 provides an illustrative schematic diagram of the method according to embodiments of the present invention wherein a transplanted organ for example including but not limited to a kidney, liver, heart, lung, graft, vascular graft and/or skin graft of a human body 10 that is treated with acoustic energy waves 110 provided with an acoustic energy generating treatment system 100. The treatment according to embodiments of the present invention is provided to prevent the onset of Delayed Graft Function (herein ‘DGF’) and/or to alleviate and/or improve symptoms associated with Delayed Graft Function (herein ‘DGF’), so as to increase the likelihood of organ implantation success.

In embodiments, the method of treatment is configured to apply acoustic energy 110, most preferably in the form of low energy shockwave treatment to a treatment area and/or region 15 comprising the transplanted organ selected from kidney, liver, heart, lung, graft, vascular graft and/or skin graft of the human body 10. Optionally the acoustic energy 110 may be provided in the form of ultrasound as detailed below. In embodiment the acoustic energy provided may be a combination of both low energy shockwaves and ultrasound energy.

In embodiments, the low energy shockwave treatment may be utilized to treat at least one or more treatment area 15 comprising an implanted organ that has undergone an organ transplant procedure, for example including but not limited to kidney, heart, liver, lung, graft, vascular graft and/or skin graft. In embodiments, the treatment is provided so as to increase likelihood of implantation success by most preferably preventing the onset of and/or advancement of DGF.

In embodiments, the method of the present invention further provides for increasing vasodilation in and around a selected treatment area 15 encompassing at least a portion of a transplanted organ, comprising at least a portion of the implanted organ for example including but not limited to at least one or more selected from kidney, liver, heart, graft, vascular graft, lung and/or skin graft.

In embodiments, the method according to embodiments of the present invention includes the delivery of acoustic energy 110, most preferably in the form of focused low energy shockwaves 110 produced with an acoustic energy generating device 102 and system 100, most preferably in the form of a shockwave generating dedicated system 102 to deliver low energy shockwaves to the treatment area 15 so as to inhibit development and/or alleviate symptoms of Delayed Graft Function (DGF) in and around the treatment area 15 of the organ transplant procedure.

The treatment area 15 may be defined along any region and/or portion along the anatomy of the implanted organ for example including but not limited to a kidney, liver, heart, graft, vascular graft, lung and/or skin graft, or the like, a non limiting example depicting such an organ, in the form of a kidney and different treatment areas thereabout is schematically shown in FIG. 4.

In embodiments, treatment with therapeutic acoustic energy of the implanted organ and/or treatment area 15 may be provided from any convenient anatomical approach for example including but not limited to prone, supine, lateral, the like or any combination thereof.

Optionally the treatment is provided with an exemplary system 100 comprising an acoustic energy generating system and/or device 102, most preferably provided in the form selected from a shockwave device and/or optionally an ultrasound device. Optionally and preferably system 100 is configured to be utilized with the guidance of a medical imagery device and/or system 104 to facilitate targeting the treatment area 15 and/or transplanted organ.

In embodiments the acoustic energy generating system 100, comprising an acoustic energy generating device 102, most preferably in the form of a shockwave generating device, provides for generating low energy shockwaves 110 that are more preferably focused, although they may optionally be non-focused. The shockwaves 110 are preferably delivered with a treatment head and/or applicator as is known in the art (not shown). Medical imagery system and/or device 104 is optionally but preferably utilized to visually facilitate directing acoustic energy generating system, preferably shockwave generating system 102 to generate and deliver shockwaves 110 to the appropriate location and/or treatment area 15 and/or focal zone 16 where treatment is to be applied, about a transplanted organ.

In embodiments, medical imagery system 104 may be utilized to determine and define a plurality of parameters for example including but not limited to the location, size and shape of the treatment area 15 and/or focal zone 16, about the transplanted organ and/or surrounding tissue.

Medical imagery system 104 may be provided in optional forms for example including but not limited to ultrasound, x-ray, computed tomography ('CT'), magnetic resonance ('MRI') or the like imaging technology and/or devices as is known and practiced in the art.

In embodiments the acoustic energy generating system 102 is optionally and preferably provided in the form of a Shockwave generating system and/or device 102 that may be provided in any form as is known in the art for example including but not limited to electrohydraulic (also referred to as spark gap), electromagnetic (also referred to as ‘EMSE’), and piezoelectric, or the like shockwave generating technology and/or devices as is known in the art. Preferably device 102 utilizes a treatment applicator and/or treatment head (not shown) to deliver the generated shockwaves to the targeted treatment area 15, 16.

Acoustic energy system 102, most preferably in the form of a shockwave generating system 102 is configured to produce low energy shockwaves 110 according to any shockwave parameters. Most preferably the shockwave parameters may for example include but are not limited to energy density (intensity), frequency, number of shockwaves, pressure, type of shockwave (focused or non-focused), the like or any combination thereof.

Most preferably the acoustic energy utilized, for example including but not limited to the form of low energy shockwaves treatment is provided so as to treat and/or prevent DGF in the newly implanted organ. In embodiments, the low energy shockwaves 110 applied to the treatment area 15 provides for preventing and/or treating different degrees of DGF by enhancing vasodilation in the implanted organ as an immediate response to the treatment and to further optionally and preferably render a long term effect by facilitating triggering of the biomolecular activity and/or signalizing pathway and/or cascade of biomolecular activity that is associated with DGF in and around the organ implantation site 15.

Optionally the applied acoustic energy, preferably in the form of low energy shockwave treatment provides for triggering the biomolecular activity in and around treatment area 15, 16 and/or of the signaling pathway that involves and/or is associated with at least one or more of the bio-molecular factors selected from the group consisting of eNOS VGF, TGFbeta; TIMP-1, FAK, SCF, HGF, that are involved with DGF signaling pathway.

In embodiments the cascade of molecular activity may further involve at least one or more of the following regulatory factors selected from the group consisting of: vascular endothelial growth factor (VEGF), fibroblast growth factors (FGF), angiopoietins (Ang), platelet-derived growth factor, angiogenin, angiotropin, hepatocyte growth factor, platelet endothelial cell adhesion molecule, angiostatin, endostatin, thrombospondin, CXC chemokines, Nitric oxide synthesis, NFkapaB activation, TNF-alpha mRNA expression, decreases the expression of MCP1, decrease expression of CD3 mRNA, and pigment epithelium.

The method according to the present invention provides for preventing the onset or progression of DGF in an implanted organ, most preferably a kidney. In embodiments the method of treatment comprises utilizing focused low energy shockwaves 110 having shockwave parameters including: an energy density from about 0.02 up to about 0.3mJ/mm2, frequency of about 2Hz.

Optionally the shockwave parameters utilized are configured to have an energy density of about 0.09 to about 0.1mJ/mm²; at a frequency of about 2Hz.

In embodiments the acoustic energy provided in the form of an ultrasound wave preferably comprising at least one or more of the following parameters: frequency of about 0.4 MHz up to about 3 MHz, energy density from about 100 mW/cm² up to about 800 mW/cm² SATA, duty cycle of about 10% up to about 44% , treatment exposure time per treatment zone selected from 0.5 minutes up to about 10 minutes, the like or any combination thereof.

A non-limiting example of an implanted organ is shown in FIG. 4, in the form of a kidney, however any organ for example including heart, liver, lung, graft, vascular graft, and/or skin graft, may be similarly treated. FIG. schematically depicts a kidney that is treated following organ transplant the treatment area 15, comprising the kidney, may be divided into a number of smaller treatment zones and/or sub-treatment zones16, also referred to as focal zones. The number of sub-treatment zones may be based on the overall size of treatment area 15 for example including but not limited to the following parameters associated with size: diameter, volume, area, and/or shape, the like or any combination thereof. Optionally the diameter, size, area, volume and/or shape of each sub-treatment zone may determine the number of shockwaves applied to the treatment sub-zone. FIG. 4 shows a non limiting example of the Kidney, however, similarly, any transplanted organ (not shown here) for example including but not limited to heart, liver, lung, graft, vascular graft, skin graft segment, may be similarly divided into a plurality of treatment zones and/or focal zones as shown in FIG. 4 with respect to a Kidney.

The number of sub-treatment zones utilized about a given transplanted organ may be based on technical specifications and/or parameters associated with the shockwave or ultrasound treatment device and in particular the treatment head and/or applicator. Optionally the number of treatment zones in a particular treatment area may be determined by the size of the treatment zone of the treatment head utilized for treatment.

FIG. 2 shows a flow chart revealing the method of treatment according to embodiments of the present invention. First in state 200 an organ transplant procedure is undertaken, optionally the organ transplant and/or tissue graft of at least one organ selected from kidney, liver, heart, lung, graft, vascular graft, and/or skin graft or the like, may utilize the method according to the present invention. Optionally the method according to embodiments of the present invention may be similarly adapted and utilized for dual or multiple simultaneous transplantation procedures wherein at least two organs are transplanted simultaneously. Embodiments of the present invention provide for treating an implanted organ and/or tissue with acoustic waveform energy for example in the form of low energy shockwaves and/or ultrasound waves.

In embodiments the shockwaves treatment protocol comprises low energy shockwaves comprising at least one or more of the following parameters: energy density of energy density from about 0.02 up to about 0.3mJ/mm2; up to 4 treatment sessions immediately following the transplant procedure; at least 1 daily treatment session for at least 21 days and up to 28 days; at least 1 and up to 40 treatment session; at least 1 and up to 3 treatment sessions daily; at least 100 shockwaves and up to 5000 shockwave in an individual treatment session; treatment focal zone having a diameter from about 2 mm and up to 50 mm; a treatment protocol of up to 4 weeks; a treatment method of up to 28 days, from about 60 shockwaves per minute up to about 360 shockwave per minute; shockwave frequency of up to about 6Hz, or any combination thereof.

In embodiments the ultrasound wave treatment protocol comprises ultrasound waves comprising at least one or more of the following parameters: frequency of about 0.4 MHz up to about 3 MHz, energy density from about 100 mW/cm² up to about 800 mW/cm² SATA, duty cycle of about 10% up to about 44% , treatment exposure time per treatment zone selected from 0.5 minutes up to about 10 minutes, the like or any combination thereof.

In embodiments, following the transplant and/or tissue graft procedure the acoustic energy may be applied within a time frame of 48 hours, more preferably within 24 hours, optionally within 12 hours, optionally within 8 hours, optionally within 4 hours, and optionally and more preferably within 2 hours, or the like.

In embodiments, the initial treatment may be provided following the transplant procedures and within 24 hours following the initial indications and/or emergence and/or development of DGF.

Next in an optional stage 201, immediately following the transplant procedure and most preferably up to about 2 hours following the end of the transplant procedure, the implanted organ may undergo up to 4 treatment sessions, wherein each treatment session comprises up to about 5000 shockwaves, and shockwave energy of up to about 0.3mJ/mm2. Optionally this immediate and/or preliminary treatment may be provided for implanted organs harvested from a deceased donor.

Next in stage 202, during the healing period following transplantation, system 100 and with the use of imaging device 104 is utilized to image the transplanted organ. Imagining device 104 preferably facilitates identifying and selecting the treatment zone(s) 15,16 about the treatment site comprising the implanted organ and perform an analysis of the focal zones and/or treatment area is provided.

Next in stage 203, the treatment zones identified in stage 202 are selected in order to properly target them with the acoustic energy generated with system 100, and in particular shockwave system 102.

Next in stage 204, the acoustic energy parameters are selected from the shockwave parameters and/or optionally the ultrasound parameters.

Next in stage 205 the selected acoustic energy is applied over the selected treatment area and/or focal zones, wherein the application of the selected acoustic energy form is delivered to the treatment site(s), wherein the treatment is provided from 1 and up to 3 times daily for at least 21 days and up to 28 days.

Finally in stage 206 the treatment is ceased if the implanted organ shows return to function within the 28 day treatment window and proper functioning.

FIG. 3 shows a schematic illustration of a non-limiting treatment protocol according to the present invention where non-invasive low energy shockwave 110 to treat an implanted organ, for example a kidney after kidney transplant the treatment area 15, or at least one or more focal zone 16. The depicted treatment is configured for an extended term treatment providing for ensuring return to function of the implanted organ and therein alleviating symptoms and/or preventing onset of DGF. A non limiting example is shown, where the treatment may be provided, for example, over a span of 6 days and may include two active treatment periods between one period of rest.

The optional treatment protocol shown calls for two active treatment sessions per day. Optionally the treatment protocol may comprise up to four active treatment session per day, and may be provided for a period of up to 28 days.

Optionally the treatment protocol may be personalized and/or configured by controlling the relative number of treatment periods and rest periods.

Optionally an active treatment period comprises a three week period that includes up to four active treatment session per day where acoustic energy in the form of at least one of ultrasound waves and/or low energy shockwaves are delivered to a targeted treatment area 15 and/or treatment zone 16.

Optionally the treatment protocol may be configured according to at least one or more parameters for example including but not limited to: length of active treatment period, number of active treatment sessions per week, length rest periods, frequency of rest period, number of shockwaves per treatment session, overall number of shockwaves delivered during active treatment period, the like or any combination thereof.

Optionally the number of active treatment sessions may be provided from about 1 session up to about 40 sessions. Optionally 12 active treatments may be provided during the treatment protocol according to the present invention. Optionally the number of active treatment session may for example be 1, or 2, or 3, or 4 or 5 or 6, or 7 or 8 or 9 or 10 or 11, or 12 or 13 or 14 or 15 or 16 or 17 or 18 or 19 or 20 or so sessions.

Optionally the duration of the treatment protocol according to the present invention may be from about 1 day up to about 28 days.

Optionally treatment may be provided periodically, continuously, sequentially, intermittently, according to a schedule comprising consecutive sessions and/or with at least one or more intersession recesses and/or rest periods. Optionally the length of the recesses and/or rest periods may vary according to the required treatment protocol.

FIG. 4 provides an illustrative schematic diagram of the implanted kidney 160 and surrounding structures including kidney 160, adrenal gland 170, nephron and glomerulus , and renal artery. FIG. 4 further provides a schematic illustration of a plurality of optional focal zones and/or treatment zones 16 depicted about the kidney 160 and kidney structures associated with the kidney.

Most preferably the low energy shockwave treatment according to the present invention is provided under the visual guidance of an imaging device or system 104 for example including but not limited to an ultrasound , CT, MRI or the like imaging technology and/or devices as is known and practiced in the art.

Most preferably imaging device and/or technology provides for aiding in defining the low energy shockwave treatment focal zone 16where treatment is to be applied for example including but not limited to the any portion of the kidney, adrenal gland 170, renal artery, the like or any combination thereof.

Optionally the shockwave treatment protocol may be focused on the glomerulus to bring about improved blood flow therethrough and associated kidney structures.

Optionally a cascade of molecular activity that may for example, involve but is not limited to at least one or more of the following bio-molecular factors selected from the group: Transforming Growth Factor beta ('TGF-β' or 'TGFbeta'); Tissue Inhibitor of MetalloProteinases (TIMP); TIMP metallopeptidase inhibitor 1 (TIMP-1); Focal Adhesion Kinase ('FAK'); Stem Cell Factor ('SCF'); Hepatocyte Growth Factor ('HGF'); Stromal Derived Cell Factor 1 ('STF-1'); Octamer-Binding Transcription Factor 4 (Oct-4); Kidney Injury Molecule-1 (KIM-1); Stromal Cell-Derived Factor 1 (SDF-1) also known as C-X-C motif chemokine 12 (CXCL12); Hypoxia Inducible Factor 1 ('HIF-1'); Norepinephrine (NE); Integrin; Beta 1 integrin; Monocyte Chemoattractant Protein-1 ('MCP-1'); mRNA of the cluster of differentiation 3 T-cell co-receptor ('CD3 mRNA'); Vascular Endothelial Growth Factor (VEGF); Fibroblast Growth Factors (FGF); Endothelial Nitric Oxide Synthase (eNOS); Angiopoietins (Ang); platelet-derived growth factor; angiogenin; angiotropin; hepatocyte growth factor; platelet endothelial cell adhesion molecule; angiostatin; endostatin; thrombospondin; Chemokine family of the form CXC ('CXC'); Nitric Oxide ('NO'); Nuclear Factor Kappa-light-chain-enhancer of activated B cells ('NFkapaB'); Tumor Necrosis Factor Alpha mRNA ('TNF-alpha mRNA'); pigment epithelium, endothelial progenitor cell or the like

Optionally the shockwave parameters utilized may be configured to have a frequency of about 2Hz and energy density from about 0.02 to about 0.3mJ/mm2. Optionally the shockwave parameters utilized are energy density of about 0.09 to about 0.1mJ/mm2; at a frequency of about 2Hz.

Optionally each treatment session may comprise up to about 5000 shockwaves. Most preferably each treatment comprises about 2400 shockwave that are delivered to the kidney structure. Optionally the number of shockwaves per treatment session may be applied to at least one and more preferably a plurality of treatment zones 164 about the kidney structures. Optionally and more preferably a plurality of zones from about 5 up to about 15 zones may be treated during a treatment session. Optionally each zone may be treated with about 100 shockwaves to about 500 shockwaves, that may be distributed amongst a plurality of zones from about 5 zones to about 15 zones forming the kidney structures.

### Example I – The treatment of Delayed Graft Function (DGF) after Kidney transplant

The treatment protocol according to the present invention may be provided to prevent or cure Graft Function (DGF) after Kidney transplant.

Preferably immediately following the kidney transplant the kidney is treated with at least 100 and up to about 2000 low energy shockwaves, and optionally up to about 5000 low energy shockwaves during an active treatment session. The low energy shockwave treatment is repeated for treatment course including at least 2 active treatment sessions following the procedure. Optionally and more preferably the active treatments continue daily from 1 and up to 3 treatments per day, thereafter with preferred 6 (six) active treatment sessions until the kidney functions are restored, for example with return of normal urine production, properly and not more than the three weeks. Optionally, the treatment could be repeated in the future according to the as directed by a physician discretion.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Citation or identification of any reference in this application shall not be construed as an admission that such reference is available as prior art to the invention.

Section headings are used herein to ease understanding of the specification and should not be construed as necessarily limiting.

What is claimed is:

1. A method for preventing or treating Delayed Graft Function (DGF) following a transplant procedure by treating a transplanted organ or tissue with acoustic energy following the transplantation and/or graft procedure.

2. The method of claim 1 wherein applying the acoustic energy over a treatment area comprising at least a portion of the transplanted organ or tissue within a 24 hour period following the transplantation.

3. The method of claim 1 wherein the acoustic energy is applied at least two hours following the end of the transplant procedure.

4. The method of claim 1 wherein the acoustic energy is applied within a 24 hour period following initial indications of DGF.

5. The method of claim 1 wherein the acoustic energy applied is provided in form of low energy shockwave treatment having shockwave parameters including: an energy density from about 0.02 up to about 0.3 mJ/mm2, at a frequency of about 2Hz; and
a) wherein the treatment protocol applies up to 5000 shockwaves over at least a portion of the transplanted organ or tissue, the treatment is characterized in that up to about 5000 low energy shockwaves are delivered to the at least a portion of the transplanted organ or tissue.

6. The method of claim 1 wherein the acoustic source of energy is in the form of ultrasound waves having at least one parameter selected from:
a) frequency of 0.4to 3 MHz; or
b) Energy density of 100 to 800 mW/cm² SATA; or,
c) 10 to 44 % duty cycle; or
d) treatment exposure time for at least 0.5 minutes and up to 10 minute per treatment area.

7. The method of claim 1 wherein the transplanted organ or tissue is selected from at least one of: kidney, liver, heart, lung, graft, vascular graft, skin graft, or a combination thereof.

8. The method of claim 7 wherein the treatment site comprising the transplanted organ or tissue is divided into a plurality of small treatment zones, wherein each treatment zone is provided with at least 100 shockwaves and up to about 500 shockwaves.

9. The method of claim 1 wherein the acoustic energy source is a combination of ultrasound wave and shockwaves.

10. The method of claim 1 wherein the acoustic energy provided is configured to achieve an immediate and/or short term effect of vasodilation in the tissue surrounding the transplanted organ or tissue.

11. The method of claim 1 wherein the treatment is applied immediately following the transplant procedures within a timeframe of from 2 hours and up to about 72 hours following the organ transplant procedure.

12. The method of claim 1 provided wherein the source of the transplanted organ or tissue is harvested from a deceased donor.

13. The method of claim 12 provided to revive vasculature of the harvested organ.

14. The method of claim 13 provided to revive collateral blood vessels of harvested organ.

15. The method of claim 1 wherein the acoustic energy is applied at least once a day for a period of up to at least 21 days following transplantation.

16. The method of claim 1 wherein the treatment is provided in up to three individual treatment sessions per day.

17. The method of claim 1 wherein the acoustic energy is administered to the transplanted organ or tissue over at least one treatment area and/or at least one or more treatment zones defined about the organ or tissue.

18. The method of claim 14 wherein the treatment area, comprising the transplanted organ or tissue, may be divided into a plurality of individual treatment zones and/or focal zones.
